(19) United States
(12) Patent Application Publication
(10) Pub. No.: US 2006/0247550 A1
(43) Pub. Date: Nov. 2, 2006

(54) System and methods for gating heart signals

(76) Inventors: Arvind Thiagarajan, Chennai (IN); Tat-Jin Teo, Sunnyvale, CA (US); Damon J. Coffman, Portland, OR (US)

Correspondence Address: Kang S. Lim, 3494 Camino Tassajara Road, #436, Danville, CA, 94506, US

(21) Appl. No.: 11/377,903

(22) Filed: Mar. 16, 2006

Related U.S. Application Data
(63) Continuation-in-part of application No. 10/477,606, filed on Nov. 14, 2003.
(60) Provisional application No. 60/668,082, filed on Apr. 5, 2005.

(30) Foreign Application Priority Data
May 28, 2001 (IN) 431/MAS/2001

(51) Int. Cl. A61B 5/02; A61B 5/05

(52) U.S. Cl. 600/528; 600/500; 600/407

(57) ABSTRACT

A heart gating system in which the heart sounds are used to generate the acquisition trigger signals. This is possible because the various heart sound components have characteristics in the temporal domain to allow for distinguishing the S1's and S2's. Hence, the individual heart sound components can be temporally distinguished and gating signals can be generated based on the identified heart sounds such as the S2 heart sound. In addition, S1 also has a frequency spectrum different from that of S2. Hence, in another embodiment of the invention, the frequency characteristics of the heart sounds can be used to distinguish S1 from S2. The combined temporal and frequency characteristics of the heart sounds can also be used to distinguish S1 from S2. In addition, detecting carotid pulses and/or changes in thoracic cavity can also used to better distinguish between S1 and S2.

### PRIORITY AND INCORPORATION BY REFERENCE

This application claims priority of U.S. Provisional Patent Application Ser. No. 60/668,082, filed Apr. 5, 2005, and titled “Method and Apparatus for Gating with Heart Sound”, which is incorporated by reference herein. This application is also a continuation-in-part application of and claims priority of U.S. patent application Ser. No. 10/477,606, having a PCT filing date of May 28, 2002, having a foreign priority date of May 28, 2001, and titled “Heart Diagnosis System”, which is also incorporated by reference herein.

### BACKGROUND OF THE INVENTION

Many imaging equipment provide good image quality of a three dimensional object when the object is stationary during the acquisition. For in-vivo imaging, cardiac cycle and respiratory motion represent two major issues. While respiratory motion can be much reduced by requiring the patient to hold the breadth, cardiac motion can only be mitigated by synchronization with the cardiac cycle. The most ubiquitous synchronization method currently is using the electrocardiogram (ECG). The QRS complex of the ECG provides an indication of the electrical event which leads to the systole phase of the cardiac cycle. The diastole phase of the heart is less distinct on the ECG as the re-polarization of the myocardium does not generate as distinctive an electrical event as the depolarization during contraction of the myocardium preceding the systole.

On the other hand, the heart sounds are very often equally distinctive in both systole (first heart sound—S1) and diastole (second heart sound—S2). These heart sounds are generated by the various heart valves and the hemodynamics of the blood moving within the chambers. Since the end of diastole phase provides the most quiescent state for the heart, the ability for heart sound to provide a synchronization signal for the diastole becomes a very desirable feature in heart sound analysis.

In many patients, various diseases cause the electrical signals to deviate (electromechanical disassociation) from those when the patient is healthy, while this may provide diagnostic value, it makes many three-dimensional imaging of the cardiac structure challenging as the ECG no longer provides the robust trigger for synchronization. Very often, the mechanical aspects of the heart remains functional and the heart sounds from these patients can provide the needed trigger information for synchronization with the cardiac cycle.

U.S. Pat. No. 5,383,231 describes a method of using ECG to synchronize the CT acquisition while U.S. Pat. No. 4,991,580 describes an improved method to synchronize MR imaging with ECG. U.S. Pat. No. 6,501,979 describes combining ECG and PPU (Peripheral Pulse) to obtain a better synchronization of MR imaging and cardiac phase. U.S. Pat. No. 6,721,386 combines ECG and cardiac mechanical signal to gate CT image acquisition. These methods all require ECG signals, solely or in conjunction with other signals, to establish the cardiac phase.

When ECG signals are used as gating signal, they suffer from the following three short-comings:

(1) they are very sensitive to the placement of the electrodes. Any errors in the placement position will affect the accuracy of the ECG signal. This will impact any subsequent processing of the ECG signal to generate the trigger signal for gating.

(2) The ECG is better at indicating the systole of the cardiac phase than the diastole of the cardiac phase. This is because the electrical event due to the depolarization of the myocardium during the systolic contraction generates a prominent QRS complex. The corresponding re-polarization of the myocardium during the diastolic relaxation phase produces a less focus T wave. However, it is the end of the diastolic phase when the heart is most quiescence and is the desired phase for the reconstruction.

(3) The time separation between the electrical QRS and the mechanical systole is also shorter than that separating T wave and diastole. Using the electrical events as a proxy for the underlying mechanical systole and diastole therefore also becomes less accurate for the diastolic phase.

Very often, a time delay is added to the time of QRS complex based on prior cardiac cycles to estimate the end of diastole for the current cardiac cycle. This will not be very accurate when the heart rate is high or not uniform. In addition, the estimate will always be somewhat inaccurate due to normal physiological variability of the electrical and mechanical synchronization of the heart cycle.

This is particular true in the case of prospective triggering where the acquisition of the diagnostic imaging equipment acquires images only upon the presence of the acquisition trigger signal. With retrospective triggering, the physiologic signals, ECG or heart sounds, are acquired continuously along with the acquisition of the images by the diagnostic imaging equipment. After all the images are acquired, the physiologic signals are then analyzed to generate the trigger signals. The images corresponding to the desired cardiac phase are then retrospectively assembled to form the gated reconstruction. In this retrospective triggering mode, any non-uniform heart rate can be detected and taken into account. However, retrospective triggering if less efficient as it takes longer time and also acquires data that may not be needed.

In some very sick patients, the electrical activities of the heart suffer from serious abnormalities. Their ECG signals can no longer be used for gating purposes. These patients very often would still have good mechanical heart sounds that can be used for gating the cardiac cycle.

U.S. Pat. No. 4,546,777 describes a gating scheme based completely on heart sound without reference to ECG. This method is applicable when the cardiac cycle data can be read out via the systole trigger signal and the diastole trigger signal, like the X-ray cardiac data described in the patent. The three-dimensional imaging equipment like MR and CT used today do not allow those imaging data to be read out based on any systole trigger signal or diastole trigger signal. The complexity of the image reconstruction excludes the possibility of sending out the raw image data upon those systole and diastole trigger signals. Instead, they rely on acquisition of ECG signal to be used internally for synchronization during the image reconstruction.

In using combination signals, U.S. Pat. Nos. 6,721,386 and 6,510,979 mentioned above both require ECG and either cardiac mechanical signal or peripheral pulse, respectively, to obtain the improved gating performance. In contrast, we describe an embodiment where the heart sounds are combined with peripheral pulse, without the use of ECG.

It is therefore apparent that a need exists for better gating the reconstruction of many of today's diagnostic imaging modalities like the CT and MR equipment.

### SUMMARY OF THE INVENTION

To achieve the foregoing and in accordance with the present invention, a method and system of gating heart sounds is provided. Such a gating system is useful for synchronizing heart imaging systems.

In one embodiment, the heart sounds are used to generate the acquisition trigger signals. This is possible because the various heart sound components have characteristics in the temporal domain to allow for distinguishing the S1's and S2's. For example, in a resting human, the temporal separation between S1 and S2 is substantially less than the separation between S2 and the next S1. Hence, the individual heart sound components can be temporally distinguished and gating signals can be generated based on the identified heart sounds such as the S2 heart sound.

In addition, S1 also has a frequency spectrum different from that of S2. Accordingly, the individual heart sounds can be distinguished using a combination or unary use of temporal or frequency band-pass filtering. Hence, in another embodiment of the invention, the frequency characteristics of the heart sounds can be used to distinguish S1 from S2. In yet another embodiment of the invention, the combined temporal and frequency characteristics of the heart sounds can be used to distinguish S1 from S2.

In some embodiments, a pulse is also used to further distinguish between S1 and S2, thereby combining the temporal and/or frequency characteristics of heart sounds with a peripheral pulse in the analysis. This is particular useful in patients where the heart rate is high and there are other confounding heart sound components that can make the determination of S1 and S2 difficult. In addition, detecting changes in thoracic cavity can also used to better distinguish between S1 and S2.

These and other features of the present invention will be described in more detail below in the detailed description of the invention and in conjunction with the following figures.

### BRIEF DESCRIPTION OF THE DRAWINGS

In order that the present invention may be more clearly ascertained, one embodiment will now be described, by way of example, with reference to the accompanying drawings, in which:

FIG. 1A is a schematic representation of a typical normal heart signal as might be collected and analyzed by the heart diagnosis system of one embodiment of the present invention;

FIG. 1B is a schematic representation of a typical heart signal as might be collected and analyzed by the heart diagnosis system of one embodiment of the present invention, the signal exhibiting the effects of heart murmurs;

FIG. 2 is a schematic diagram of an exemplary data collection device for one embodiment of a heart sound gating system in accordance the present invention;

FIG. 3 is a schematic representation of the frequency response of the sensor of the data collection device of FIG. 2;

FIG. 4 is a schematically view of one embodiment of the chest piece for the data collection device of FIG. 2;

FIGS. 5A and 5B are twin perspective views of the chest piece of FIG. 4;

FIG. 6 is a schematic depiction of the process by which various voltages are provided by the one power supply in the sensor of the data collection device of FIG. 2;

FIG. 7 is a schematic circuit diagram for the circuit of the amplifier of the data collection device of FIG. 2;

FIG. 8 is a view of the pin assignment of the IC of the amplifier of the data collection device of FIG. 2;

FIG. 9 is a pin diagram for the IC of the amplifier of the data collection device of FIG. 2;

FIG. 10 is a timing diagram of the ADC of the data collection device of FIG. 2;

FIG. 11 is an internal diagram of the ADC of the data collection device of FIG. 2;

FIG. 12 is a connection diagram for the clock generator IC of the data collection device of FIG. 2;

FIG. 13 depicts the pin assignments of the UART of the data collection device of FIG. 2;

FIG. 14 is a flow diagram of the full signal processing performed by the data collection device of FIG. 2;

FIG. 15A is a plot of a notional original signal to be processed by the collection device of FIG. 2;

FIG. 15B is a plot of a hard thresholding signal corresponding to the signal of FIG. 15A;

FIG. 15C is a plot of a soft thresholding signal corresponding to the signal of FIG. 15A;

FIG. 16A is a heart signal before de-noising;

FIG. 16B is the heart signal of FIG. 16A after de-noising by the data collection device of FIG. 2;

FIG. 17 is a plot a filtered signal (top) and a signal energy envelope (bottom);

FIG. 18 is a dendrogram obtained in the cluster analysis of a heart signal;

FIG. 19 is a flow diagram summarizing the procedure for detecting murmurs;

FIG. 20 is a plot of the heart signal, which includes S1, S2, systole and diastole, before extraction of S1 and S2;

FIG. 21 is a plot of the signal of FIG. 20 after the extraction of S1 and S2;

FIG. 22 is a plot of the energy envelope of the early diastole;

FIG. 23 is a plot of a heart sound signal exhibiting continuous murmur;

FIG. 24 is a plot of the signal of FIG. 23 following the calculation and smoothing of its Shannon's energy envelope;

FIG. 25 is a plot of a heart signal waveform exhibiting splits;

FIG. 26 is a plot of the signal of FIG. 25 following the calculation and smoothing of its Shannon's energy envelope;

FIG. 27 is a schematic representation of murmur relative to other portions of the heart cycle;

FIG. 28 is a block diagram illustrating an exemplary implementation in which one embodiment of the heart sound gating system of the present invention generates a gating trigger signal for a heart imaging system, the gating system using input from a heart sound acquisition device place near the heart;

FIG. 29 illustrates a typical ECG waveform, a typical heart sound waveform, and a typical carotid pulse; and

FIG. 30 shows the frequency spectra of the various heart sounds as well as the corresponding time intervals.

### DETAILED DESCRIPTION OF THE INVENTION

The present invention will now be described in detail with reference to several embodiments thereof as illustrated in the accompanying drawings. In the following description, numerous specific details are set forth in order to provide a thorough understanding of the present invention. It will be apparent, however, to one skilled in the art, that the present invention may be practiced without some or all of these specific details. In other instances, well known process steps and/or structures have not been described in detail in order to not unnecessarily obscure the present invention. The features and advantages of the present invention may be better understood with reference to the drawings and discussions that follow.

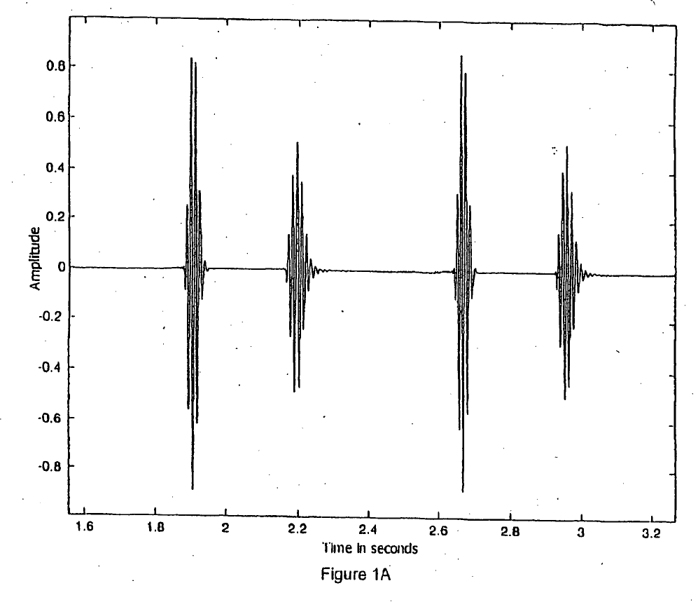

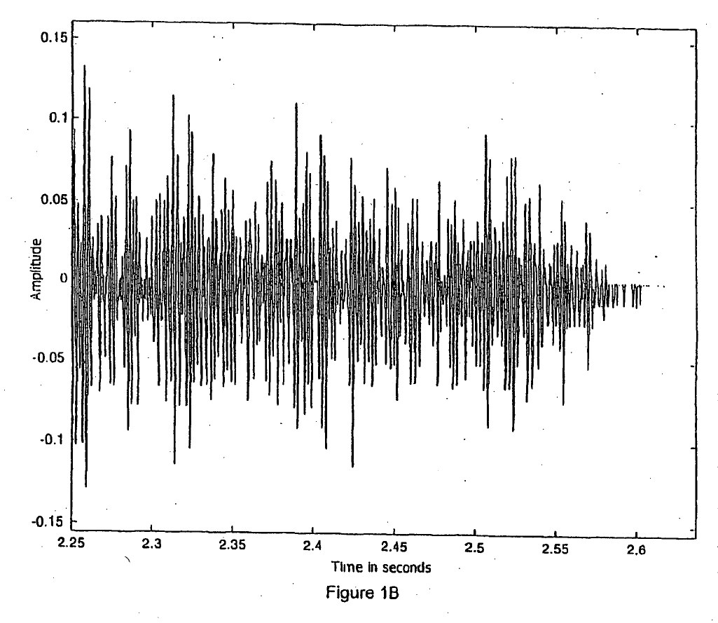

FIG. 1A is a schematic representation of a typical normal heart signal; FIG. 1B is a similar representation of a heart signal from a heart with murmurs. These signals are typical of the sound signals collected by the present system. Heart sounds typically lie in the frequency range of 20 to 1 kHz, with S1 approximately in the range 80 to 150 Hz, S2 approximately in the range 60 to 100 Hz, S3 approximately in the range of 30 to 60 Hz, S4 approximately in the range of 20-40 Hz, and heart murmurs anywhere in the range 20 Hz to 1 kHz.

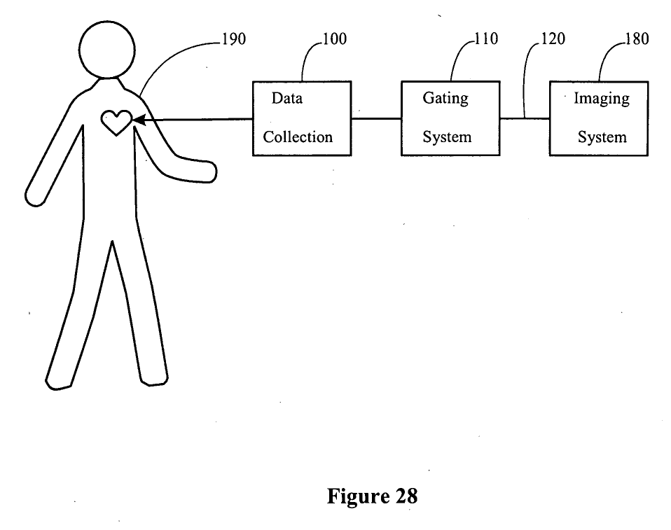

To facilitate discussion, FIG. 28 is a block diagram showing an exemplary implementation in which a heart sound gating system 110 generates a gating trigger signal for a heart imaging system 180. A human 190 is coupled to a heart data collection device 100. In this example, device 100 incorporates a heart sound acquisition device.

Collection device 100 provides electronic heart sounds to gating system 110. Gating system 110 in turn provides a gating trigger signal 120, which in this example is a pseudo ECG signal 120, for the ECG input of imaging system 180, such as an MR or CT equipment. Collection device 100 can be an electronic stethoscope such as a Thinklabs Rhythm Stethoscope commercially available from Thinklabs, Inc., Cantennial, Colo. (www.thinklabsmedical.com).

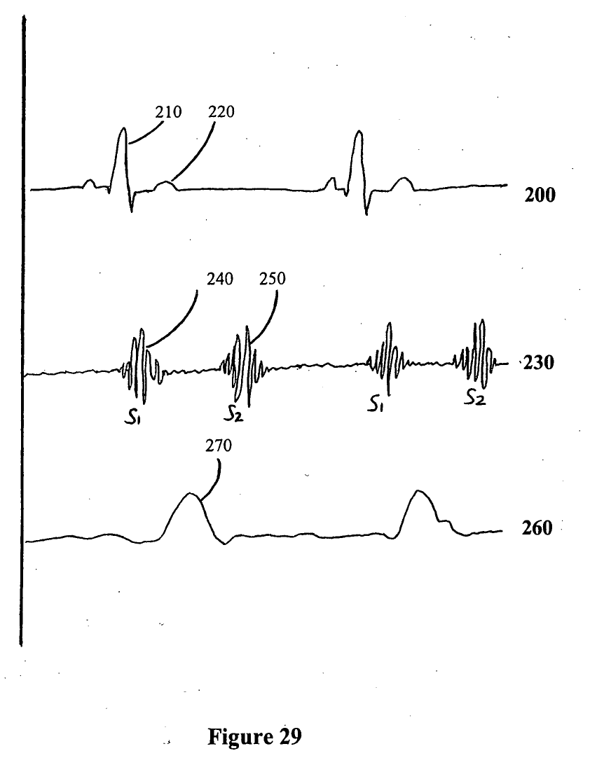

In FIG. 29, upper trace 200 illustrates a typical ECG waveform. The QRS complex, waveform 210, precedes the systole phase of the heart when the ventricles contract to send the blood out of the heart into the systemic circulation and pulmonary circulation. It is apparent that the T wave, waveform 220, which denotes the re-polarization phase of the heart muscle, hence an indication of diastole phase, is much less distinct compared with the QRS complex which characterizes the depolarization of the systolic contraction of the myocardium

Middle trace 230 of FIG. 29 illustrates a typical heart sound waveform. Waveform 240 denotes the first heart sound (S1) indicating systole, while waveform 250 denotes the second heart sound (S2) indicating diastole.

In one embodiment of the invention, the heart sounds are used to generate the acquisition trigger signals. As is apparent from FIG. 29, the various heart sound components have characteristics in the temporal domain to allow for distinguishing the S1's and S2's. For example, when human 190 is resting, the temporal separation between S1 and S2 is substantially less than the separation between S2 and the next S1, typically, 1/3 to 2/3 ratio in normal resting rhythm. With this observation, we can distinguish the individual heart sound components with standard tools of signal processing. The relative success of this approach is highly dependent on the ability to acquire high quality heart sound signals that are free from external artifacts or other physiological but non-heart related sounds generated by human 190.

As discussed above, in addition to the time separation from S1 to the following S2 being shorter than that from S2 to the next S1, heart sound S1 also has a frequency spectrum different from that of heart sound S2. For example, in a typical adult, S1 is approximately in the range 80 to 150 Hz, and S2 approximately in the range 60 to 100 Hz. With this observation, we can distinguish the individual heart sound components with standard tools of signal processing, such as a combination or unary use of temporal or frequency band-pass filtering. Hence, in another embodiment of the invention, the frequency characteristics of the heart sounds can be used to distinguish S1 from S2 by filtering heart sounds above and below a suitable threshold frequency, e.g., 100 Hz. In yet another embodiment of the invention, the combined temporal and frequency characteristics of the heart sounds can be used to distinguish S1 from S2. The relative success of this approach is also highly dependent on the ability to acquire high quality heart sound signals free from external artifacts or other physiological but non-heart related sounds generated by human 190.

Referring again to FIG. 29, lower race 260 illustrates a typical carotid pulse. Waveform peak 270, is shown having an arrival time with respect to the heart sounds in trace 230. This peak 270 can therefore also be used to distinguish between S1 and S2. Accordingly, in another embodiment of the invention, the temporal and/or frequency characteristics of heart sounds can be combined with a peripheral pulse in the analysis. This is particular useful in patients where the heart rate is high and there are other confounding heart sound components that can make the determination of S1 and S2 difficult if only the heart sounds are utilized. From the relative arrival time of S1 and the carotid pulse, one can increase the robustness of S1 determination.

For those skilled in the art it is clear that the carotid pulse is not the only method for assisting the separation of S1 from S2. For example, patent application PCT/AU2004/000465 describes one possible method for measuring changes (volume, blood flow, or heart wall motion in combined or unary modes) within the thoracic cavity using microwave backscatter. Thus, another embodiment of the invention can incorporate such a methodology for correlation with heart sounds separate the S1 and S2 intervals. In yet a further embodiment of the invention the combination and simultaneous analysis this type of signal(s) can assist in determining with high accuracy the specific onset and offset of various hearts sounds and cycles, such as the S1, S2 and the systolic and diastolic interval.

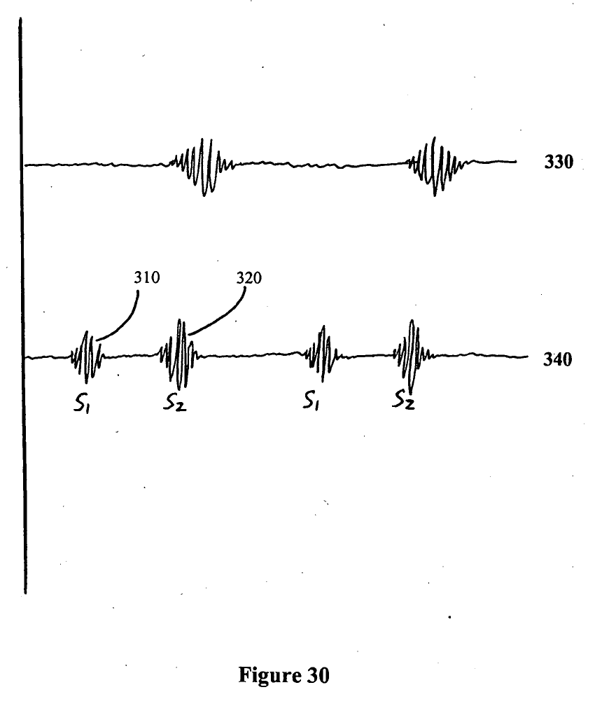

FIG. 30 illustrates the frequency bands of the various heart sounds as well as the corresponding time intervals. Upper trace 330 shows the heart sounds above 125 Hertz, which happens to be the predominant frequencies of murmurs, in this example, diastolic murmurs. Lower trace 340 shows the heart sounds in the 0 to 125 Hertz range, with waveform 310 showing the lower frequency range of S1 and waveform 320 showing the lower frequency range of S2. As shown in FIG. 30, the frequency content of heart sounds S1, S2 and the frequency of diastolic murmurs are quite different, with minimal S1, S2 energy appearing in higher frequency range as illustrated by traces 330, 340. Hence, by eliminating, e.g. by filtering, the higher frequencies associated with murmurs, the heart sounds S1 and S2 can be better distinguished from each other.

Once the S1's and S2's are identified, a canonical ECG signal can be constructed that mimics what one can expect from a normal and healthy heart. It is this pseudo ECG signal that is presented to the downstream diagnostic imaging system 180 as illustrated in FIG. 28. This pseudo ECG signal satisfy the expectation of the diagnostic imaging system 180 thereby allowing for plug and play compatibility with the gating requirement of imaging system 180.

If imaging system 180 allows for other form of trigger signals, they can be easily accommodated as is apparent to one of ordinary skill in interfacing to imaging system 180. For example, heart imaging system 180 could be designed to trigger on either S1 only, S2 only, or on both S1 and S2, thus giving a greatly superior method for reconstructing diagnostic images. In addition, pseudo ECG signal can be similar in waveform to a real ECG signal or be a simplified digital signal.

Once the S1's are identified, this a-priori knowledge can assist with the identification of other heart sound components, including S2's.

In accordance with another aspect of the present invention, the identification of mummers with its higher frequency content and where it occurs during the cardiac cycle, also help with the S1 and S2 identification.

This is also true with other heart sound components like ‘splits’, S3 and S4, as other physiological information can be incorporated in the heart sound analysis to increase the identification of S1 and S2, as is well known to one of ordinary skill in the physiology of auscultation.

Other modifications to the invention as also possible. For example, the use of specific frequency spectra information via spectral analysis of the signals in addition to or separate from band-pass information can provide methodology for separation and identification of heart sound components.

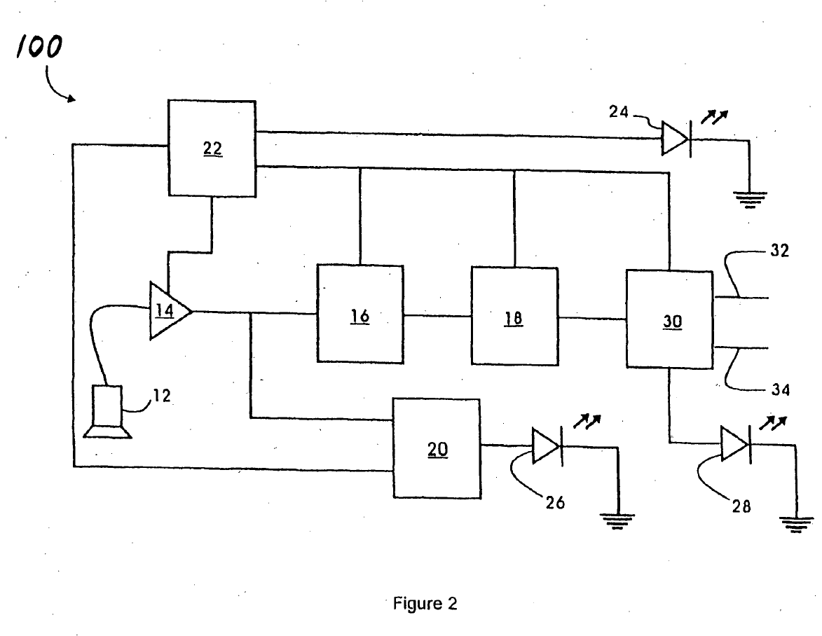

Referring back to FIG. 2, which is a schematic diagram illustrating one embodiment of a heart data collection device 100 in accordance with the present invention. The data collection device 100 is connectable to a personal computer (also a part of the system); the data collection device includes a transducer in the form of sensor 12 for detecting the weak sounds between 20 Hz and 1 kHz made by the heart (sufficient for diagnosis of most diseases).

The sensor 12 receives the sound from the vibration of a diaphragm (not shown) provided adjacent to the sensor. The data collection device 100 converts the signals into electrical signals by means of the sensor 12, then amplifies the electrical signal by means of amplifier 14, removes unwanted noise, digitizes the signal by means of analog to digital converter (ADC) 16 and converts the signal to RS232 format for transmission to the computer. The device 100 uses the D9 serial port of the computer.

The device operates on a power supply in the form of single 9 V alkaline battery 22, and has a D9 male serial port to attach the serial port cord for the PC, one on/off switch to switch on/off the main power supply, and a three pin stereo connector to connect the sensor 12 to the amplifier 14. The AC mains are not used since they introduce a hum into the signal corresponding to the frequency of the mains (50 Hz in many countries), and medical equipment should be precise as possible and free from noise. The power supply 22 provides the voltages required by the device 100 (i.e. +5 V and −5 V to the amplifier 14, 1.5 V for the sensor 12 and 5 V for the digital section).

The device 100 also has a universal asynchronous receiver transmitter 18, a comparator 20 and three LEDs:

a) orange LED 24: glows whenever power is switched on;

b) red LED 26: glows flickeringly whenever the sensor is properly receiving sound signals from the heart, the flicker indicative of the heart signal; and

c) green LED 28: glows when data is being transmitted to the computer.

Thus, as soon as the power is switched on orange LED 24 glows, indicating that the power is going to the hardware. Based on the intensity of the light glowing, the battery level will be known to the user. If the LED 24 glows low then it is time to change the battery 22. The signals are acquired from the hardware only when a signal comes from the computer. When this signal comes from the computer green LED 28 glows indicating that a signal is ready to be acquired. Once green LED 28 glows, the acquired signals are displayed in the monitor of the computer. The red LED 26 flickers according to the heart sounds. This LED 26 indicates whether the gain is sufficient or whether the gain has to be increased, and flickers owing to the signals coming out of a comparator 20. The amplified signals are sent to the comparator 20, whose first input is set at a standard voltage (viz. that of power supply 22) and whose second input receives the amplified signal. Whenever the signal level is above the preset value the red LED 26 flickers.

There is one switch for gain adjusting. This switch can be used if it is found that the signal being acquired by the computer is not of the expected level. This switch is used to reduce the gain if it is observed that the signal is clipped, and to increase the gain if the signal is very feeble (indicated by the red LED's not flickering).

Owing to the vibrations occurring on the diaphragm, sound is produced. This sound is picked up by the sensor. The sensor used is a condenser microphone which requires a supply of 1.5 V from the circuit itself. The sensor 12 is selected to give a good electrical signal based on the signal impinging on it.

The intensity of the sounds will vary from person to person. A thin person, for example, will have a high sound intensity, while for an overweight person the signals will have weak intensity. The signal must therefore be amplified to a optimum level by means of amplifier 14. The amplifier's basic functionality is to amplify the signal given to it in a ratio of the gain set by the user. This amplified signal has certain advantages, such as reduced noise and an increased signal strength lying in the input range of the ADC 16.

The ADC 16 converts the input analog signal into a digital signal using successive approximation registers commonly known as SARs. SAR ADCs are reliable and economical.

The ADC output is input into the UART 18. The UART's main function is to convert the signal from the ADC 16 into asynchronous or RS232 standard for subsequent transmission to the computer at a specified baud rate set for data processing.

If we speak or listen to music it is because the intensity of the sound is good enough to create disturbances in the medium and hence make an impression on our ears and a sound is heard.

As alluded to above, the device 100 also a serial port 30 for sending signals to (32) and receiving signals from (34) the PC.

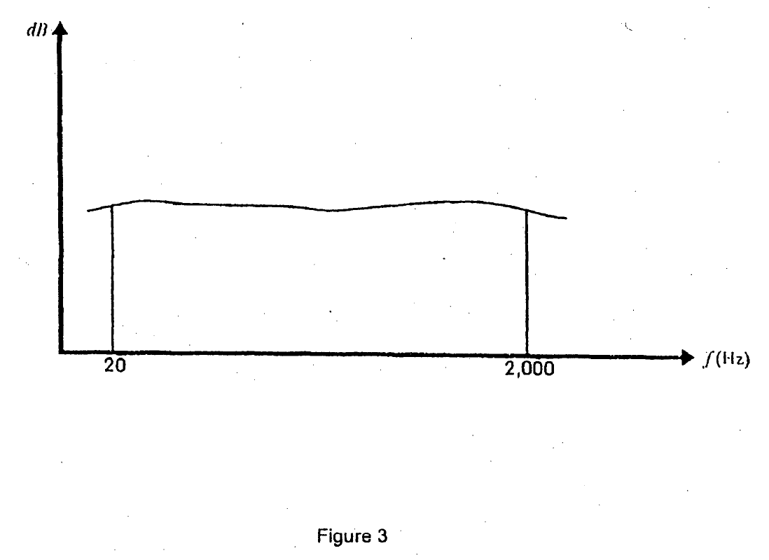

The intensity of heart sounds is very low, so various factors should be considered in the design of the sensor 12 of the present system. These factors include frequency response, voltage output of the sensor, unidirectionality, cost and availability. The sensor 12 should have consistent frequency response in the required frequency range, namely 20 Hz to 2 kHz. In other words the sensor should be able to pick up all the frequencies in the said range and should be able to give out similar response to all the frequencies. FIG. 3 is a schematic representation of the frequency response of the sensor 12 of the present system, plotted as amplitude against frequency.

The output of the sensor 12 is measured in volts/db/pascal, that is, the voltage per unit of intensity per unit pressure. Since the heart sounds are of very low intensity, the sensor 12 is able to detect the heart sounds and produce a strong output.

The sensor 12 is unidirectional in the sense that sound coming from one direction alone are converted to electrical signals and output. Its rejects—as far as possible—sounds coming from other directions.

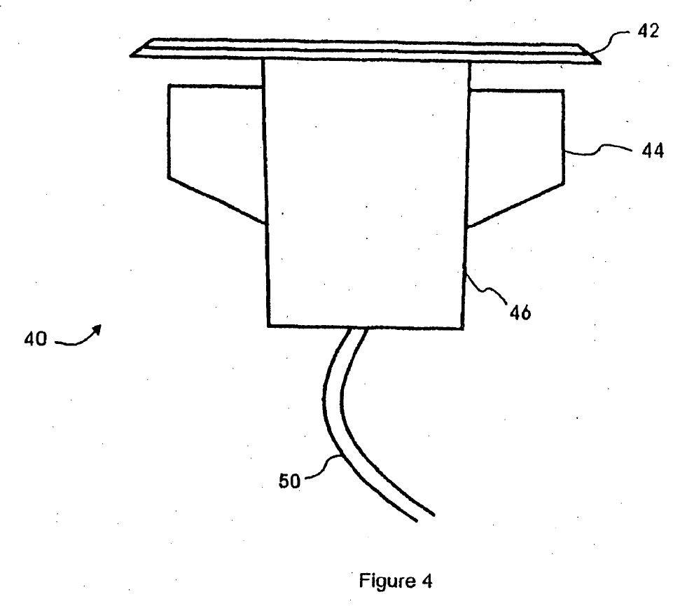

The sensor 12 and its diaphragm are located in a chest piece, shown schematically at 40 in FIG. 4, which is similar in appearance to a stethoscope sensor, so patients are not disquieted by the use of unfamiliar equipment. The diaphragm 42 filters out high frequency noises, and the chest piece 40 is designed to prevent physical contact between the sensor 12 (within generally cylindrical portion 44) and the diaphragm 42. Further, the chest piece includes a sound damper between the sensor 12 and the chest piece walls, and is designed to be fixable to the patient (such as with a strap around a patient's upper body) by means of a buckle 46, so that—unlike a stethoscope—the examining person does not have to hold the chest piece in place.

Output cable 50 is also shown.

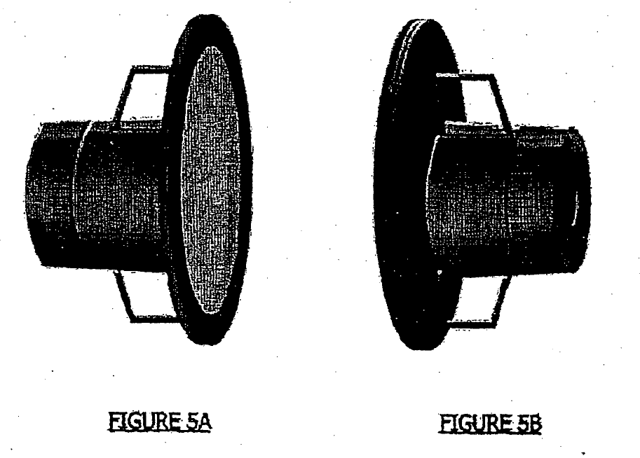

FIGS. 5A and 5B are twin perspective views of the chest piece 40, shown from two different angles. In FIG. 5A, the forward (patient contact) face of the diaphragm 42 is visible.

As mentioned above, the power supply 22 is in the form of a 9 V battery. As +5 V, −5 V, −9 V are required for the components used in the device 100, ICs with +9 V, −9 V, +5 V and −5 V outputs are employed.

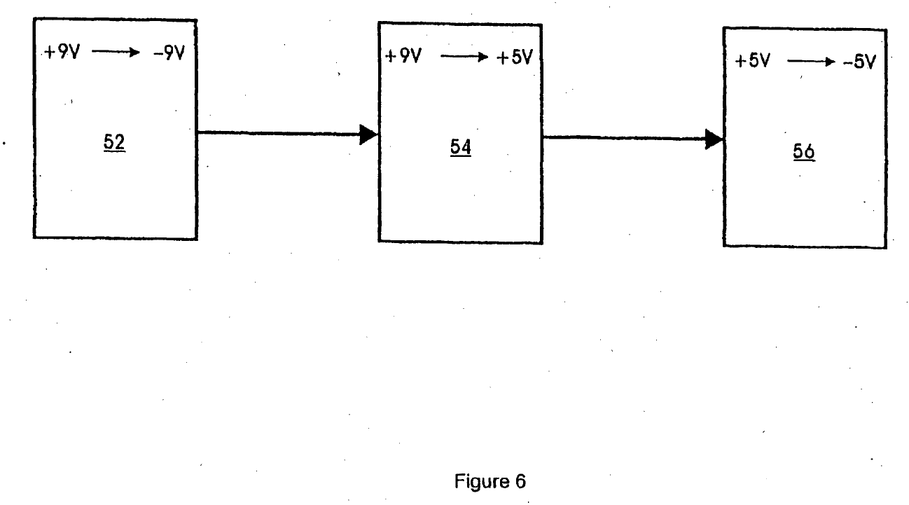

The +9 V is made into +5 V using a voltage regulator and then given to the IC which has +5 V and −5 V outputs. The voltage converter IC is a charge pump converter: it uses a capacitor as a ‘bucket’ to pump charge from one place to another. Referring to FIG. 6, in this case the IC connects the positive terminal of first capacitor 52 to +9 V from the battery and its negative terminal to ground. First capacitor 52 charges up to 9 V from the battery 22. This IC then connects the positive terminal of first capacitor 52 to ground, and the negative terminal to pin 5. This lets first capacitor 52 dump the charge into second capacitor 54. The negative terminal of second capacitor 54 is tied to pin 5, so it gets a negative voltage equal to the voltage across first capacitor 52.

This charge pumping is a very efficient way to convert voltages. The only power lost is that power which is dissipated in the resistance of the switches inside the IC and the series resistance of the capacitors, as well as the power to run the internal oscillator that flips the switches when needed.

By itself, the IC runs at about 7 to 10 kHz, so there will be ripple of that amount on the output of second capacitor 54 and on the +9 V output from the battery 22 also. Audio equipment that uses this voltage could have an audible whine. However, the IC has a frequency boost feature. If pin 1 is connected to the power supply 22, the oscillator frequency goes up by about 6:1. The oscillator then works well above the audio region so that any whine will be inaudible.

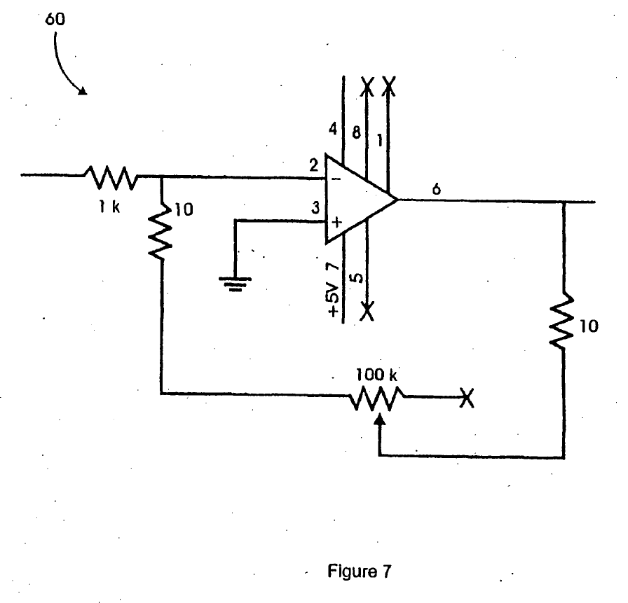

FIG. 7 is a schematic circuit diagram for the circuit 60 of amplifier 14. The circuit 60 provides a low noise transformer-less amplifier, with a true-balanced circuit, sensor powering and high common mode rejection ratio. This design also includes sensor input loading of 1 k.OMEGA. Input loading is capacitive reactive at higher frequencies to attenuate unwanted RF and ultrasonic signal at the input terminals. Sensor powering circuit provides power for sensor that require 1.5 V.

The signals coming from the sensor 12 are weak and have to be amplified. This is done using the amplifier circuit 60. The signals coming from the sensor are amplified based on the resistor combination. The amplifier design includes a gain switch in the form of potentiometer. As the potentiometer is adjusted the value of resistance changes and hence the gain increases or decreases based on the movement on the potentiometer. This gain can be adjusted based on the display in the monitor of the computer.

The amplifier used here has a good flat frequency response from 20 Hz to 2 kHz. The noise voltage at 1 kHz is 4 nV/sqrt(Hz) and the noise current at 1 khz is 0.4 pA/sqrt(Hz). The unity gain bandwidth of this amplifier is 10 MHz with a common mode rejection ratio of 100 db. It has a slew rate of 13 V/.mu.s. It operates over a wide supply range of 3 V to 22 V.

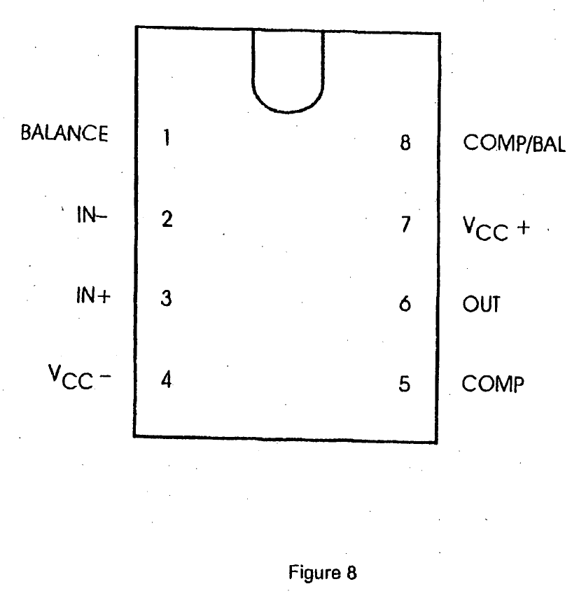

FIG. 8 is a view of the pin assignment of the amplifier IC.

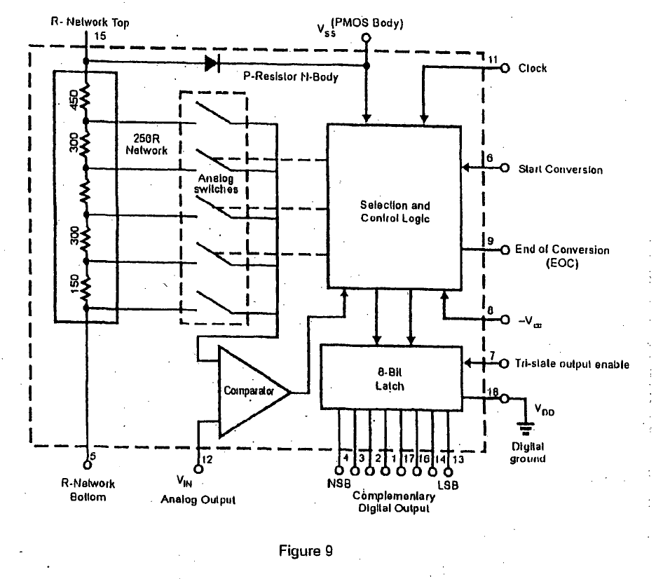

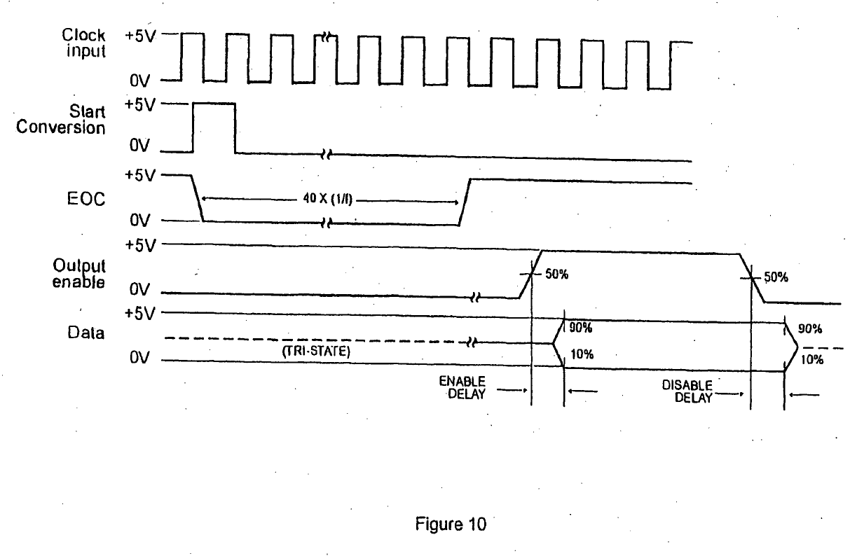

The ADC 16 has an input range of .+−.5 V, and a parallel interface. In order to meet this specification, the ADC is selected to have a conversion time of 47 clock cycles in free running mode. The pin diagram of the IC is as shown in FIG. 9, and the timing diagram of the ADC is shown in FIG. 10.

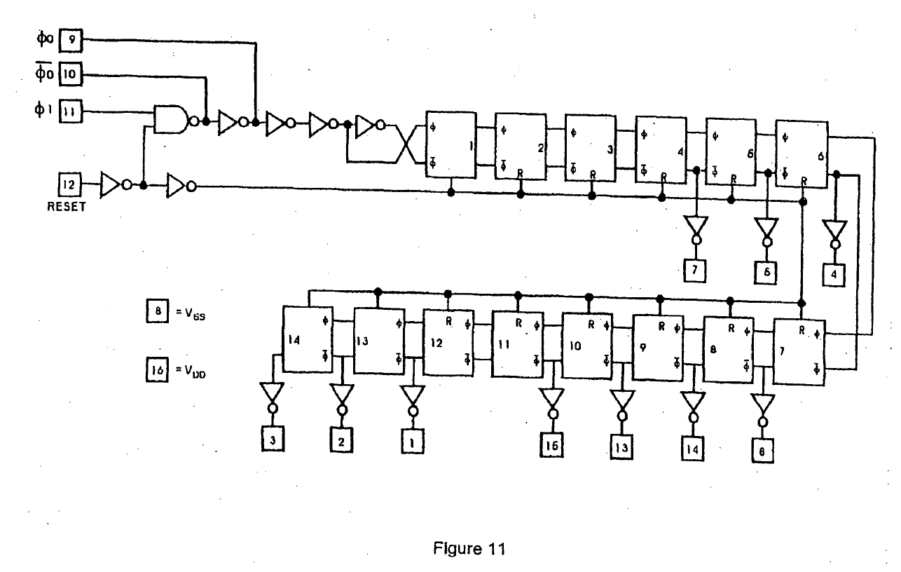

The ADC is placed in free running mode which gives the End of Conversion pulse after 47 clock cycles and starts the next conversion. The output of the ADC is an 8 bit which is a 256 combination output. The ADC clock is set to 270 kHz which is suitable to transfer the maximum of 2 kHz input signal. The clock calculation is derived from standard baud rate 57600. For 57600 baud rate, 5760 samples are transferred from UART 18 to the PC. In order to get the 5760 samples the ADC clock frequency is set to 270 kHz, that is, 5760.times.44. The ADC clock is derived from clock divider IC4060, which is a binary counter. The internal diagram of the ADC IC is shown in FIG. 11.

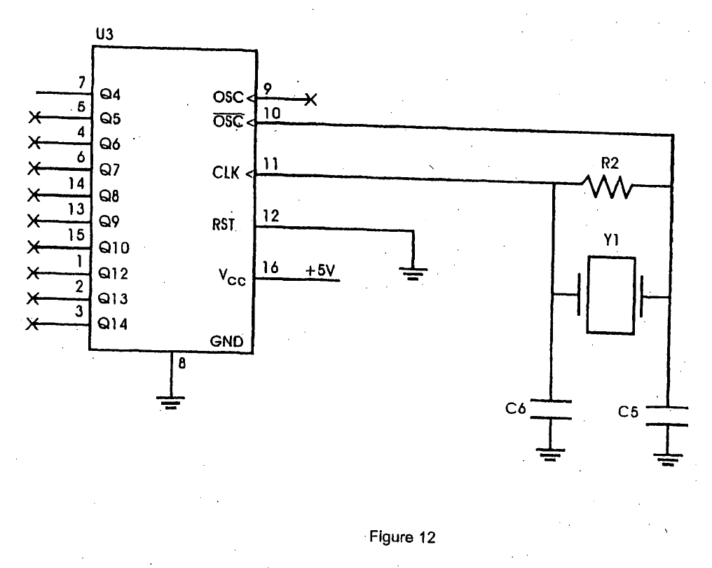

To obtain the ADC clock 270 kHz, a crystal frequency of 270 kHz multiplied by 16 (giving 4.3 MHz) is used. The connection diagram of the clock generator IC is as shown in FIG. 12.

The specification for serial ports, as used in this system, is provided in the EIA (Electronics Industry Association) RS232C standard. It states many parameters, including:

1. A ‘Space’ (logic 0) will be between +3 and +25 V;

2. A ‘Mark’ (Logic 1) will be between −3 and −25 V

3. The region between +3 and −3 volts is undefined;

4. An open circuit voltage should never exceed 25 V (in Reference to GND); and

5. A short circuit current should not exceed 500 mA.

The driver should be able to handle this without damage.

Serial ports come in two sizes: D-Type 25 pin connectors and D-Type 9 pin connectors. Both are male on the back of the PC, so a female connector is used on the peripheral device. Table 1 lists pin connections for the 9 pin and 25 pin D-Type connectors.

TABLE 1
D Type 9 Pin and D Type 25 Pin Connectors

| D Type-25 Pin | D Type-9 | | |
|---|---|---|---|
| No. | Pin No. | Abbreviation | Full Name |
| Pin 2 | Pin 3 | TD | Transmit Data |
| Pin 3 | Pin 2 | RD | Receive Data |
| Pin 4 | Pin 7 | RTS | Request To Send |
| Pin 5 | Pin 8 | CTS | Clear To Send |
| Pin 6 | Pin 6 | DSR | Data Set Ready |
| Pin 7 | Pin 5 | SG | Signal Ground |
| Pin 8 | Pin 1 | CD | Carrier Detect |
| Pin 20 | Pin 4 | DTR | Data Terminal Ready |
| Pin 22 | Pin 9 | RI | Ring Indicator |

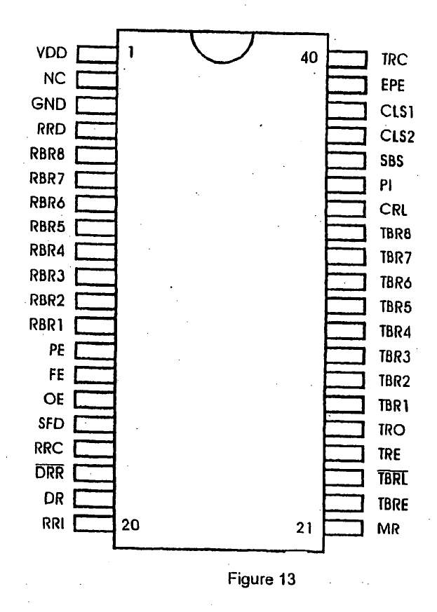

The UART's Control Register is made up of Parity Inhibit (PI), Stop Bit Select (SBS), Character Length Select (CLS1 and 2) and Even Parity Enable (EPE). These inputs can be latched using the Control Register Load (CRL) or if this pin is tied to high, changes made to these pins will immediately take effect. The pin assignments of the UART 18 are shown in FIG. 13, and listed in Table 2.

TABLE 2
Pin Description for UART

| PIN | ABBR. | FULL NAME | NOTES |
|---|---|---|---|
| 1 | VDD | +5 V Supply Rail | |
| 2 | NC | Not Connected | |
| 3 | GND | Ground | |
| 4 | RRD | Receiver Register Disable | When driven high, outputs RBR8:RBR1 are |
| | | | High Impedance |
| 5:12 | RBR8, RBR1 | Receiver Buffer Register | Receiver's data bus |
| 13 | PE | Parity Error | When High, a parity error has occurred. |
| 14 | FE | Framing Error | When High, a framing error has occurred, i.e. the |
| | | | stop bit was not a logic 1. |
| 15 | OE | Overrun Error | When High, Data has been received but the |
| | | | nData Received Reset had not yet been activated |
| 16 | SFD | Status Flag Disable | When High, Status Flag Outputs (PE, FE, OE, |
| | | | DR, and TBRE) are High Impedance. |
| 17 | RRC | Receiver Register Clock | x16 Clock input for the Receiver Register. |
| 18 | nard | Data Received | Active Low. When low, sets Data received |
| | | | Output Low (i.e. Clears DR) |
| 19 | DR | Data Received Reset | When High, data has been received and placed |
| | | | on outputs RBR8:RBR1 |
| 20 | RRI | Receiver Register | RXD - Serial Input. Connect to Serial Port, Via |
| | | | RS 232 receiver. |
| 21 | MR | Master Reset Register | Resets the UART. UART should be reset after |
| | | | applying power. |
| 22 | TBRE | Transmitter Buffer | Empty when High, indicates that transmitter |
| | | Register | buffer register is empty, thus all bits including the |
| | | | stop bit have been sent. |
| 23 | nTBRL | Transmitter Buffer | Active Low. When low, data present on TBR8:TBR1 |
| | | Load/Strobe | is placed in Transmitter Buffer Register. A |
| | | | low to High Transition on this pin, then sends the |
| | | | data. |
| 24 | TRE | Transmitter Register | When High, Transmitter Register is Empty, thus |
| | | Empty | can accept another byte of data to be sent. |
| 25 | TRO | Transmitter Register Out | TXD - Serial Output. Connect to Serial Port, via |
| | | (TXD) | RS-232 Transmitter. |
| 26:33 | TBR8:TBR1 | Transmitter | Data Bus, for Transmitter. |
| | | Buffer Register | Places Data here. |
| 34 | CRL | Control Register Load | When High, Control Register (PI, SBS, CLS2, |
| | | | CLS1, EPE) is Loaded. Can be tied high, so |
| | | | changes on these pins occur instantaneously. |
| 35 | PI | Parity Inhibit | When High, no Parity is Used for Both Transmit |
| | | | and Receive. When Low, Parity is Used. |
| 36 | SBS | Stop Bit Select | A High selects 2 stop bits. (1.5 for 5 Character |
| | | | Word Lengths) A Low selects one stop bit. |
| 37:38 | CLS2:CLS1 | Character Length | Selects Word Length. 00 = 5 Bits, 01 = 6 Bits, |
| | | Select | 10 = 7 Bits and 11 = 8 Bits. |
| 39 | EPE | Even Parity Enable | When High, Even Parity is Used, When Low, |
| | | | Odd Parity is Used. |
| 40 | TRC | Transmitter Register Clock | 16x Clock input for Transmitter. |

The clock divider IC has Q4 to Q14 available for use as they have external connections. This means higher Baud Rates are not obtainable from common crystals, such as the 14.31818 MHz. The UART requires a clock rate 16 times higher than the Baud Rate you will be using. A baud rate of 57600 bps, for example, requires an input clock frequency of 921.6 kHz.

The CMOS UART can handle up to 200 kbps at 5 V, but the level converter may be limited to 120 kbps, which is still within range. In PC maximum available standard baud rate is 115200; the next available baud rate is selected to be 56700.

Signal Processing

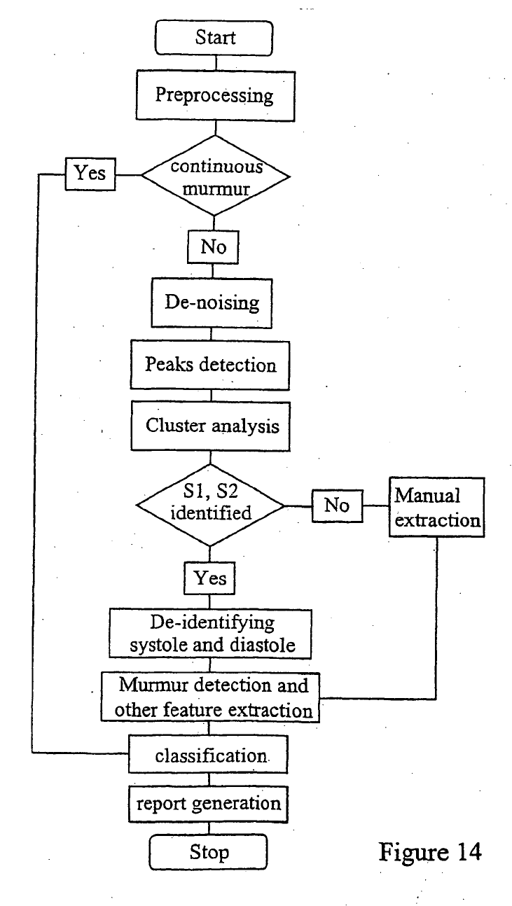

The collected signal includes noise, motion artifacts, breathing sounds and other background sounds. In order to correctly identify the actual heart sounds, the systole and diastole regions are first identified. By finding the first and seconds heart sounds the systole and diastole regions can be found. FIG. 14 is a flow diagram of the full signal processing performed by the system of the present embodiment, as is described in detail below.

The heart sounds contain frequency components from 20 Hz to 2 kHz with much of its frequency components below 1 kHz. The signals are sampled at 7200 Hz. Since the signal is sampled at a high frequency, the signal contains much redundant information. According to the Nyquist criterion, it is sufficient to sample a signal at twice the maximum frequency component present. In the present case, therefore, it is sufficient to sample the signal at 4 kHz. If the raw signal is not down sampled, the processing time will be significantly higher, so the signal is down sampled to 4 kHz.

The system supports the file formats WAV (Windows PCM Wav Format), AU and MAT (Matlab MAT file).

Since the intensity of the heart beat is variable, the signal amplitude is normalized to between +1 and −1. Thus, during preprocessing the raw signal is converted to a 4 kHz normalized signal. The signal is then available in the form of a matrix suitable for further processing.

The first and second heart sounds have their energies concentrated in the 30 to 150 Hz region. Unfortunately motion artifacts and background noise fall in essentially the same frequency range. Consequently, it is difficult to remove the noise by conventional noise removal techniques so wavelet based techniques are employed in the present system.

The general de-noising procedure involves three steps. Firstly, a wavelet is chosen and the signal is decomposed to N levels. Secondly, for each level from 1 to N, a threshold is selected and applied to the detail coefficients. Thirdly, the wavelet reconstruction is computed using the original approximation coefficients of level N and the modified detail coefficients of levels from 1 to N.

The ‘hard’ threshold signal is x if .vertline.x.vertline.>t, and is 0 if .vertline.x.vertline.<=t. The ‘soft’ threshold signal is sign(x)(.vertline.x.vertline.−t) if .vertline.x.vertline.>t and is 0 if .vertline.x.vertline.<=t.

Hard thresholding is the usual process of setting to zero the elements whose absolute values are lower than the threshold. Soft thresholding is an extension of hard thresholding, in which the elements whose absolute values are lower than the threshold are first set to zero, and then the nonzero coefficients are shrunk towards 0.

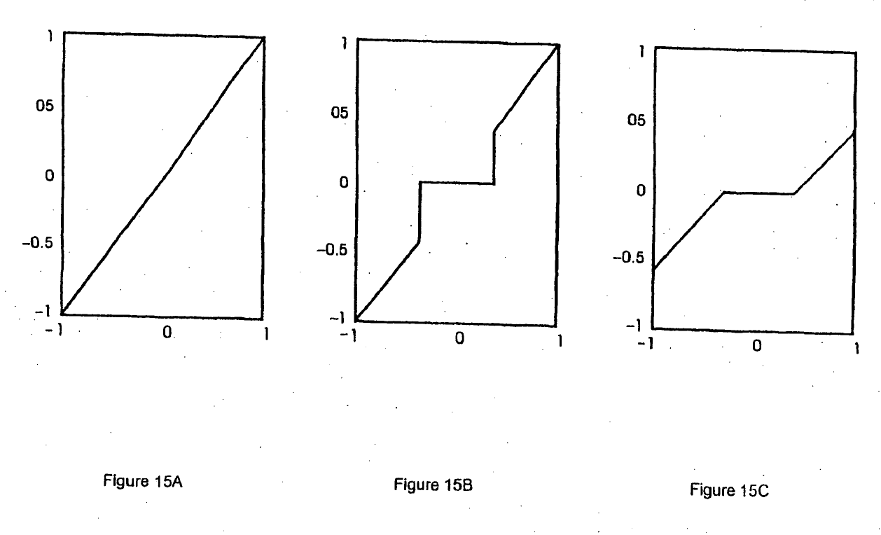

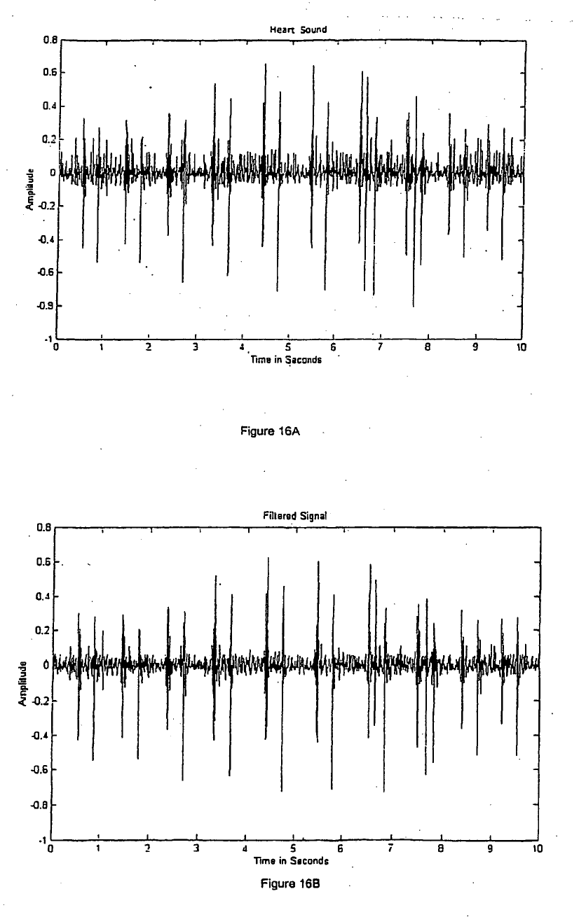

FIG. 15A illustrates a notional original signal; the corresponding hard thresholding signal is shown in FIG. 15B, and the corresponding soft thresholding signal is shown in FIG. 15C.

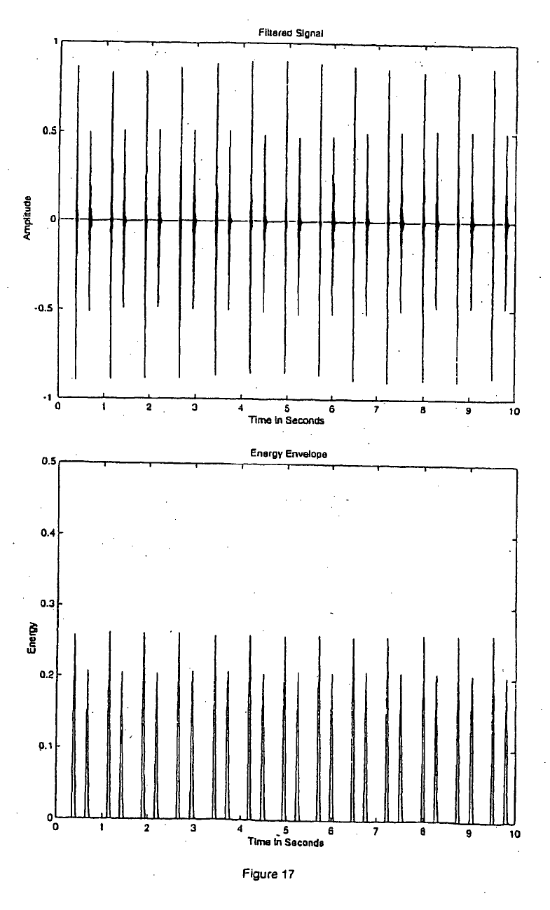

In choosing the threshold rules one can afford to lose the information contained in the murmur frequencies, as the principal aim is to enhance the first and second heart sounds (S1 and S2) to enable their successful extraction. Consequently, the decomposition levels that do not contribute to the first and second heart sounds are neglected when framing the threshold rules, and the output of the de-noise module is the raw input waveform whose first and second heart sounds are enhanced with all other unwanted components removed. FIGS. 16A and 16B are, respectively, a heart signal before and after de-noising plotted in each case as amplitude versus time.

After de-noising the prominent heart sounds are identified, which involves identifying the peaks in the signal. The peaks are regions where the amplitude of the signal is high. It is generally not possible to identify the peaks directly from the signal as they contain high amplitude oscillations. However, the peaks can be identified by filtering the signal and then calculating its envelope. The latter is done by calculating the signal's Shannon's energy is calculated, which clearly amplifies the peaks while suppressing other regions.

FIG. 17 includes plots of a filtered signal (top: amplitude versus time) and signal energy envelope (bottom: energy versus time).

The maximum amplitude of the signal is calculated for every one second of the envelope signal. The values of the envelope signal above certain percentage of the maximum value are separated. These values represent the peaks with zero values between them. Then the starting and ending point of these peaks are identified.

After prominent peaks have been identified, the following peak parameters are calculated:

1. Maximum value;

2. Area of the peak;

3. Width of the peak;

4. Starting point of the peak;

5. Ending point of the peak; and

6. Distance to the successive peak.

The cluster analysis of the peaks is performed based on the following peak parameters:

1. Maximum Amplitude of the peak;

2. Width of the peak;

3. Area of the peak; and

4. Distance to the successive peak.

Cluster analysis has been found to eliminate false peaks due to motion artifacts and breathing sounds that have escaped the de-noising process; the former are random and give rise to dissimilar peaks, and are readily eliminated by cluster analysis. Breathing sounds may produce false peaks with a higher degree of similarity, but it has been found that the maximum amplitude or width of such peaks have a low degree of similarity when compared to peaks of the first and second heart sounds, so are also eliminated by cluster analysis.

The method proceeds by:

1. Finding the similarity or dissimilarity between every pair of objects in the data set;

2. Grouping the objects into a binary, hierarchical cluster tree; and

3. Determining where to divide the hierarchical tree into clusters.

To find the similarity or dissimilarity, the distance between objects is calculated, in one of a variety of ways. In the present system, the aim is to calculate the Euclidean distance between objects in a data set of m objects, or pairs m(m−1)/2 pairs of objects. The result of this computation is commonly known as a similarity matrix (or dissimilarity matrix). In a real world data set, variables can be measured against different scales; here each of the parameters has a different amplitude. All the values in the data set are converted to the same proportional scale. At the end of this step the distance between every pair of objects is found.

In this case the ‘distance to the successive peak’ is the important parameter. This parameter shows a very high degree of similarity for the peaks due to the first and second heart sounds, owing to the fact that systole and diastole periods are relatively constant and systole period is always lesser than the diastole period. This being the case, it is reasonable to assume that the distance between successive S1 peaks and S2 peaks forms two clusters with a high degree of similarity. If there is a recurring third heart sound it will form another cluster.

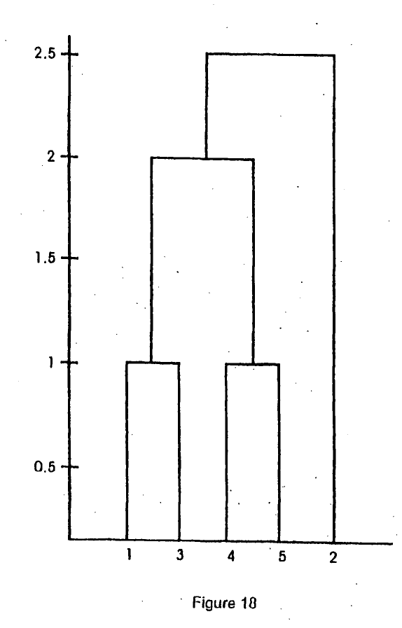

To group the objects, pairs of objects that are in close proximity are linked together using the linkage function. Once the proximity between objects in the data set has been computed, it is possible to determine which objects in the data set should be grouped together into clusters, using the linkage function. The linkage function takes the distance information and links pairs of objects that are close together into binary clusters (clusters made up of two objects). The linkage function then links these newly formed clusters to other objects to create bigger clusters until all the objects in the original data set are linked together in a hierarchical tree. The hierarchical, binary cluster tree created by the linkage function is most easily understood when viewed graphically as a dendrogram, as shown in FIG. 18; the horizontal axis represent the indices of the objects in the original data set. The links between objects are represented as upside down U-shaped lines. The height of the U indicates the distance between the objects. For example, the link representing the cluster containing objects 1 and 3 has a height of 1.

In determining where to divide, the linkage function uses the distance information generated in step 1 to determine the proximity of objects to each other. As objects are paired into binary clusters, the newly formed clusters are grouped into larger clusters until a hierarchical tree is formed in the hierarchical cluster tree, the data set may naturally align itself into clusters. This can be particularly evident in a dendrogram diagram where groups of objects are densely packed in certain areas and not in others.

The inconsistency coefficient of the links in the cluster tree can identify these points where the similarities between objects change. In our program the after finding the distance information, the inconsistency coefficient is calculated. Then the objects are grouped in to clusters.

In the typical data set there may be one, two or more groups. If the signal has S1 and S2 alone, the two natural clusters may be formed. If the signal includes other heart sounds then there may be more than 2 clusters. The inconsistent function gives the inconsistency values for each links. By setting the maximum value of the inconsistent matrix as threshold the natural divisions in the data set can be identified. If the peaks cannot be grouped, the system software indicates that automatic extraction is not possible and that manual extraction is performed.

After identifying the different groups in the peaks, the peaks are identified as S1, S2 or other heart sounds based on the previously estimated parameters; for example, S1 generally has a shorter ‘distance to successive peak’ than S2. If the signal has first, second and any third heart sounds, each of the three sounds will be grouped as three separate clusters. The heart sounds may be S3, S4, ejection click, opening snap, pericardial rub, tumor plops. Each of these sounds will differ in at least any one of the above mentioned parameters. By considering these four parameters each group can be identified. In this way all the groups are identified. Of course, the systole and diastole regions include the first and second heart sounds, but for the present purposes the systole region is taken to be the region between the end of S1 and the beginning of S2, the diastole region the region between the end of S2 and the beginning of the next S1.

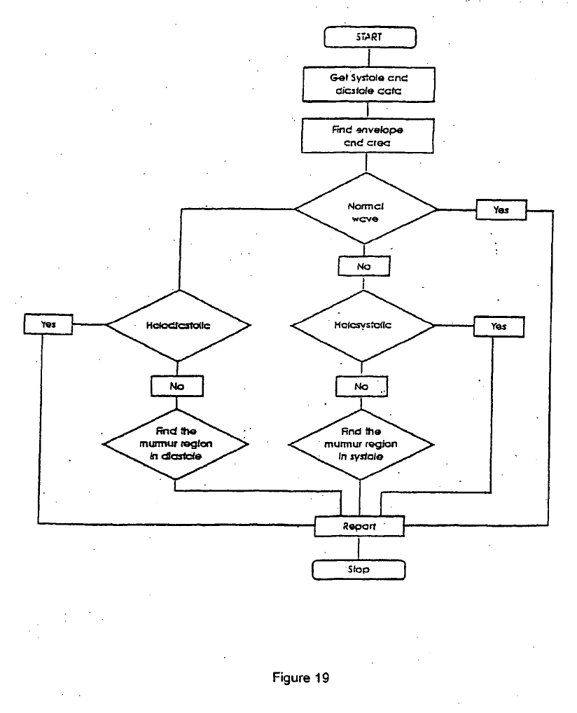

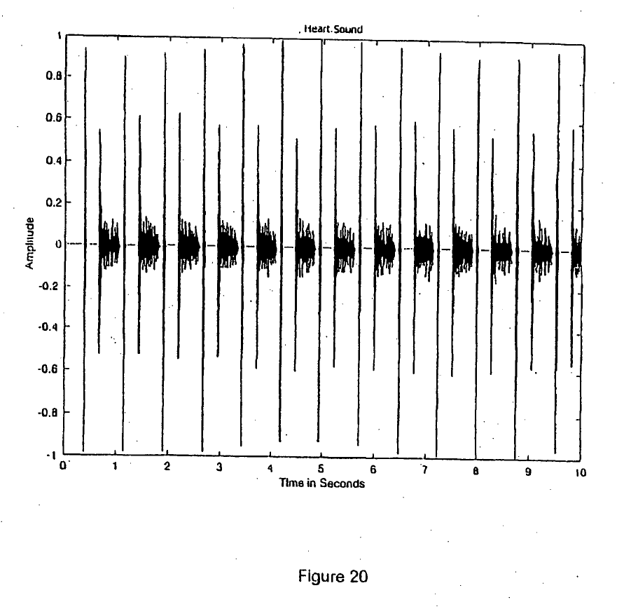

The systole and diastole data is analyzed after the extraction of S1 and S2 heart sounds from the sound signal. The procedure for detecting murmurs is summarized in the flow diagram shown in FIG. 19.

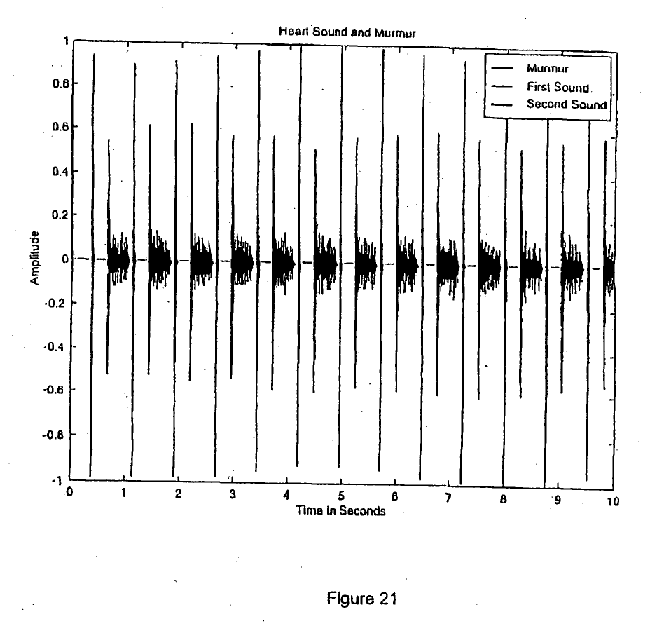

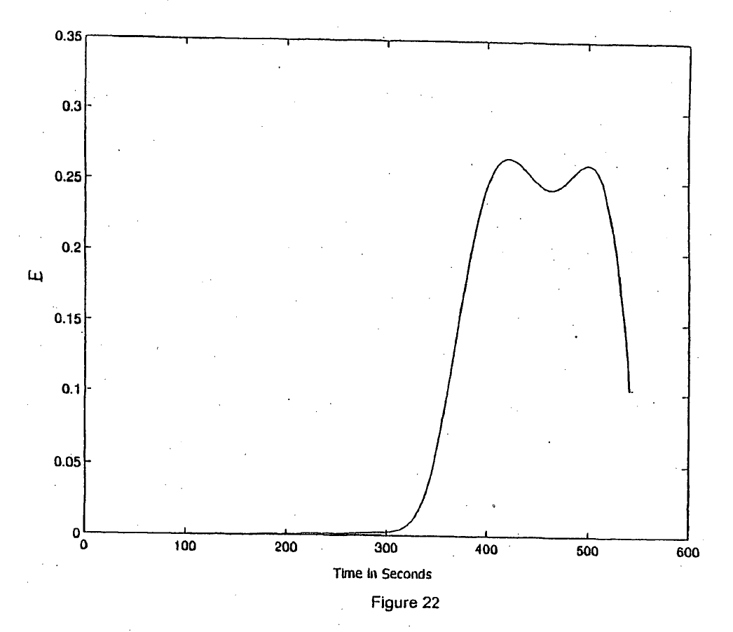

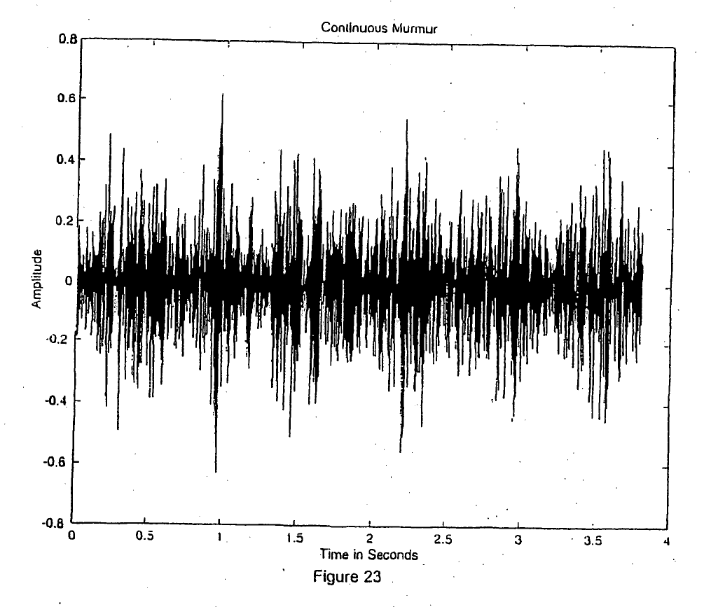

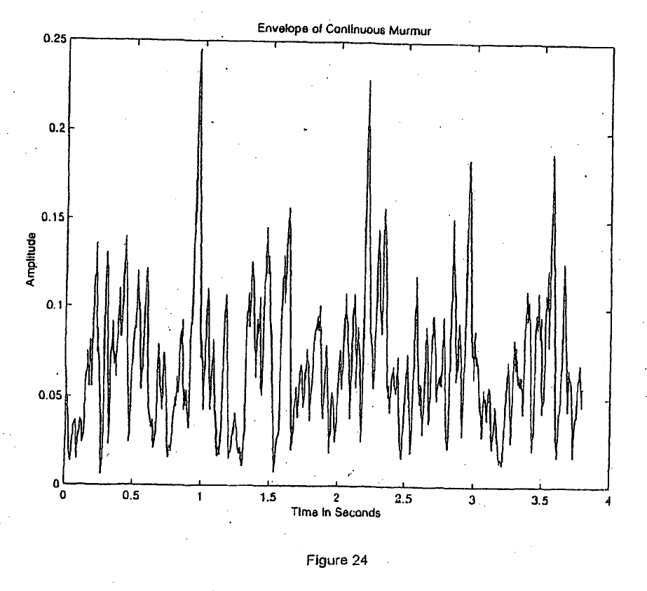

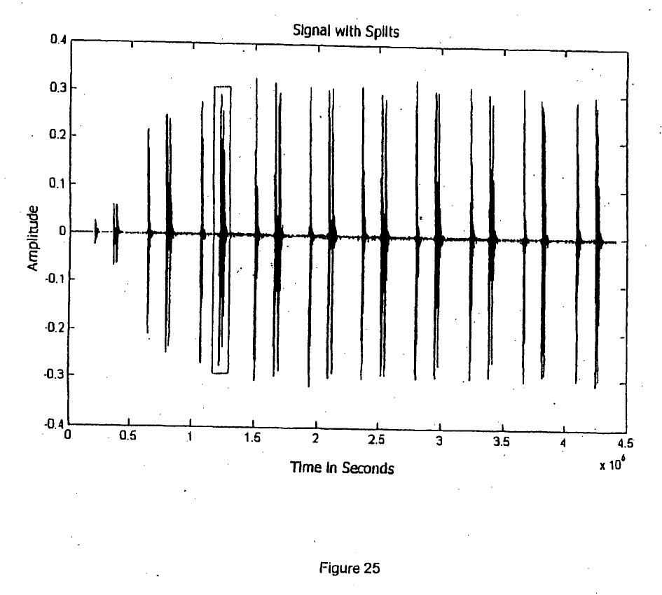

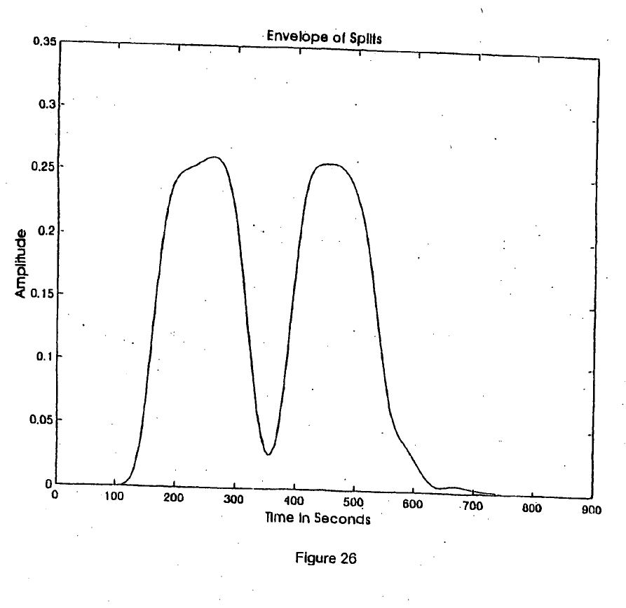

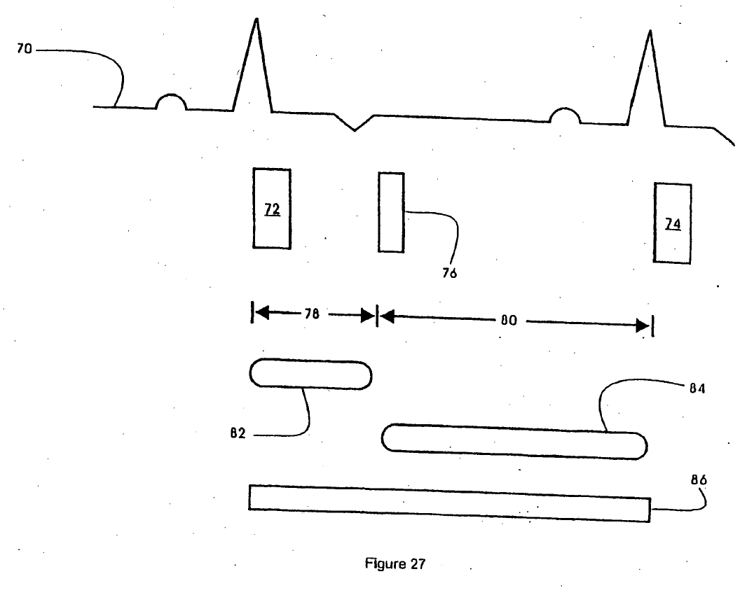

FIG. 20 is a plot of the signal, which includes S1, S2, systole and diastole. The signal after the extraction of S1 and S2 is shown in FIG. 21, in which S1 is visible as the peaks of greatest amplitude and S2 of second greatest amplitude; the murmur regions are the broader regions attached to and to the right of the S2 peaks. Next, the systole and diastole are divided into three regions each. The Shannon's energy envelope is taken for systole and diastole region to reduce the noise and enhance the signal by removing the redundant and unwanted data. A moving average smoothing operation is performed to smooth the envelope. In FIG. 21, the diastole region shows the presence of murmur. The envelope of the early diastole is shown in FIG. 22, plotted as energy E versus time t(s).

While the present invention has been described with reference to particular embodiments, it will be understood that the embodiments are illustrative and that the invention scope is not so limited. In addition, the various features of the present invention can be practiced alone or in combination. Alternative embodiments of the present invention will also become apparent to those having ordinary skill in the art to which the present invention pertains. Such alternate embodiments are considered to be encompassed within the spirit and scope of the present invention. Accordingly, the scope of the present invention is described by the appended claims and is supported by the foregoing description.

What is claimed is:

1. A method of generating heart gating signals, useful in association with a heart imaging device, the method comprising:
receiving at least two heart sounds including an S1 heart sound and an S2 heart sound;
identifying the S2 heart sound; and
sending an S2 gating signal corresponding to the S2 heart sound to the heart imaging device.

2. The method of claim 1 wherein identifying the S2 heart sound includes differentiating temporally between the S1 heart sound and the S2 heart sound.

3. The method of claim 2 wherein differentiating temporally includes measuring an S1-to-S2 interval between the S1 heart sound and the S2 heart sound, and measuring an S2-to-S1 interval between a subsequent pair of S2 and S1 heart sounds.

4. The method of claim 1 wherein identifying the S2 heart sound includes detecting a pulse.

5. The method of claim 4 wherein the pulse is at least one of a carotid pulse, a radial pulse and a brachial pulse.

6. The method of claim 1 wherein identifying the S2 heart sound includes differentiating spectrally between the S1 heart sound and the S2 heart sound.

7. The method of claim 6 wherein differentiating spectrally includes detecting a lower frequency band associated with the S2 heart sound and detecting a higher frequency band associated with the S1 heart sound.

8. The method of claim 7 wherein the lower frequency band is substantially below 100 Hertz and the higher frequency band is substantially above 100 Hertz.

9. The method of claim 1 wherein the S2 gating signal is a pseudo ECG signal which compensates for time delay between an ECG signal and the S2 heart sound.

10. The method of claim 1 wherein identifying the S2 heart sound includes detecting changes in thoracic cavity caused by at least one of volume change, blood flow and heart wall motion.

11. The method of claim 1 wherein identifying the S2 heart sound includes filtering a heart murmur sound.

12. A heart gating system, useful in association with a heart imaging device, the system comprising:
an acoustic sensor configured to receive at least two heart sounds including an S1 heart sound and an S2 heart sound;
a heart sound detector configured to identify the S2 heart sound; and
a gating signal generator configured to send an S2 gating signal corresponding to the S2 heart sound to the heart imaging device.

13. The gating system of claim 12 wherein identifying the S2 heart sound includes differentiating temporally between the S1 heart sound and the S2 heart sound.

14. The gating system of claim 13 wherein differentiating temporally includes measuring an S1-to-S2 interval between the S1 heart sound and the S2 heart sound, and measuring an S2-to-S1 interval between a subsequent pair of S2 and S1 heart sounds.

15. The gating system of claim 12 further comprising a sensor configured to detect a pulse.

16. The gating system of claim 15 wherein the pulse is at least one of a carotid pulse, a radial pulse and a brachial pulse.

17. The gating system of claim 12 wherein identifying the S2 heart sound includes differentiating spectrally between the S1 heart sound and the S2 heart sound.

18. The gating system of claim 17 wherein differentiating spectrally includes detecting a lower frequency band associated with the S2 heart sound and detecting a higher frequency band associated with the S1 heart sound.

19. The gating system of claim 18 wherein the lower frequency band is substantially below 100 Hertz and the higher frequency band is substantially above 100 Hertz.

20. The gating system of claim 12 wherein the S2 gating signal is a pseudo ECG signal which compensates for time delay between an ECG signal and the S2 heart sound.

21. The gating system of claim 12 further comprising a sensor configured to detect changes in thoracic cavity caused by at least one of volume change, blood flow and heart wall motion.

22. The gating system of claim 12 further comprising a filter for filtering a heart murmur sound.
